(19) United States
(12) Patent Application Publication
(10) Pub. No.: US 2018/0071031 A1
(43) Pub. Date: Mar. 15, 2018

(54) ALTERNATIVE ANATOMIC REGISTRATION FOR NAVIGATION

(71) Applicant: Biomet Manufacturing, LLC, Warsaw, IN (US)

(72) Inventors: Keith R. Berend, Columbus, OH (US); John R. White, Winona Lake, IN (US)

(21) Appl. No.: 15/561,642

(22) PCT Filed: Mar. 25, 2016
(86) PCT No.: PCT/US2016/024240

§ 371 Date: Sep. 26, 2017

Related U.S. Application Data
(60) Provisional application No. 62/138,447, filed on Mar. 26, 2015.

(51) Int. Cl. A61B 34/20

(52) U.S. Cl. CPC A61B 34/20; A61B 17/1739; A61B 2034/2068; A61B 2034/2055

(57) ABSTRACT

Various systems, devices, and methods for facilitating efficient anatomical registration in surgical navigation are disclosed. Such systems, devices, and methods can provide for relatively rapid collection of individual physical space data corresponding to a plurality of surface points during a dynamic data collection mode of a detection unit. Additionally or alternatively, such systems, devices, and methods can provide for collection of a plurality of data at a single point collection mode of a detection unit. The system can have a data collection unit and a registration probe. A distal portion of the registration probe can have an engagement tip. The engagement tip can be a dynamic engagement tip or a functional engagement tip.

### CLAIM OF PRIORITY

This application claims the benefit of U.S. Provisional Patent Application Ser. No. 62/138,447, filed on Mar. 26, 2015, the benefit of priority of which is claimed hereby, and which is incorporated by reference herein in its entirety.

### FIELD

The present disclosure relates to anatomic registration probes and associated systems and methods.

### BACKGROUND

This section provides background information related to the present disclosure, which is not necessarily prior art.

Surgical navigation is commonly used for various types of orthopedic surgeries. Surgical navigation systems typically require registration to the patient's anatomy. One common method of anatomical registration employs a stylus having a locator disposed on one end in conjunction with a detection unit in order to sequentially record anatomical landmarks. Because human anatomy can be geometrically complex, it is often necessary for a stylus to register hundreds or thousands of points in order to adequately define relevant patient anatomy.

### SUMMARY

This section provides a general summary of the disclosure, and is not a comprehensive disclosure of its full scope or all of its features.

The present inventors have recognized that the process of sequentially recording individual anatomical points is arduous and time consuming. Devices, systems, and methods for facilitating more efficient anatomical registration would therefore be desirable. Accordingly, the present teachings provide for a system for efficient anatomical registration. The system includes a data collection unit and a registration probe. The registration probe comprises a registration probe body that can have a distal portion, intermediate portion, a proximal portion, and a longitudinal axis defined by the intermediate portion. The registration probe can further comprise a dynamic engagement tip operably coupled to a distal end of the distal portion. The dynamic engagement tip can be rotatable with respect to the registration probe. The registration probe can also comprise a locator coupled to the proximal portion. In operation, the dynamic engagement tip of the registration probe can be placed in continuous contact with and moved around a selected anatomical area during a selected time period where the data collection system collects physical space data corresponding to individual surface points at a data collection rate. The data collection unit can determine at least one of a plurality of point-based registrations and distance between individual surface points based on the collected physical space data.

The present teachings also provide for a method for efficient anatomical registration. The method comprises: activating a data collection unit for a selected time period; placing a dynamic engagement tip of a registration probe into contact with a selected anatomical area, wherein the registration probe has a distal portion, an intermediate portion, a proximal portion, and a longitudinal axis defined by the intermediate portion, wherein the dynamic engagement tip is operably coupled to a distal end of the distal portion, and wherein the dynamic engagement tip is rotatable with respect to the registration probe; moving the registration probe relative to the selected anatomical area via rotation of the dynamic engagement tip; recording physical space data corresponding to individual surface points at a data collection rate during the selected time period; determining at least one of a plurality of point-based registrations and distance between individual surface points based on the collected physical space data.

The present teachings further provide for another device for efficient anatomical registration. The device can comprise a registration probe body and a functional engagement tip. The registration probe can have a distal portion, intermediate portion, a proximal portion, and a longitudinal axis defined by the intermediate portion. The functional engagement tip can be coupled to a distal end of the distal portion. The functional engagement tip can further comprise a contact surface geometry that can be complementary to a selected anatomical area. The device can also comprise a locator coupled to the proximal portion. In operation, the functional engagement tip of the registration probe can facilitate registration of at least one of the size, location, and orientation of the selected anatomical area.

The present teachings further provide for another method for efficient anatomical registration. A method comprising: activating a data collection unit; placing a functional engagement tip of a registration probe into contact with a selected anatomical area, wherein the registration probe has a distal portion, an intermediate portion, a proximal portion, and a longitudinal axis defined by the intermediate portion, wherein the functional engagement tip is coupled to a distal end of the distal portion, and wherein the functional engagement tip comprises a contact surface geometry that is complementary to a selected anatomical area; and recording at least one of the size, location and orientation of the functional engagement tip to facilitate registration of at least one of the size, location, and orientation of the selected anatomical area.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 illustrates an anatomical registration system;

FIG. 2 illustrates one aspect of a registration probe;

FIG. 3 illustrates another aspect of a registration probe;

FIG. 4 illustrates another aspect of a registration probe;

FIG. 5 illustrates another aspect of a registration probe;

FIG. 6 illustrates another aspect of a registration probe;

FIG. 7 illustrates another aspect of a registration probe; and

FIG. 8 is a flow chart illustrating an exemplary process of anatomical registration.

Corresponding reference numerals indicate corresponding parts throughout the several views of the drawings.

### DETAILED DESCRIPTION

Example embodiments will now be described more fully with reference to the accompanying drawings.

The present teachings provide for various systems, devices, and methods for facilitating efficient anatomical registration in surgical navigation. Such systems, devices, and methods can provide for relatively rapid collection of physical space data corresponding to individual surface points during a dynamic data collection mode of a detection unit. Additionally or alternatively, such systems, devices, and methods can provide for collection of a plurality of data at a single point collection mode of a detection unit.

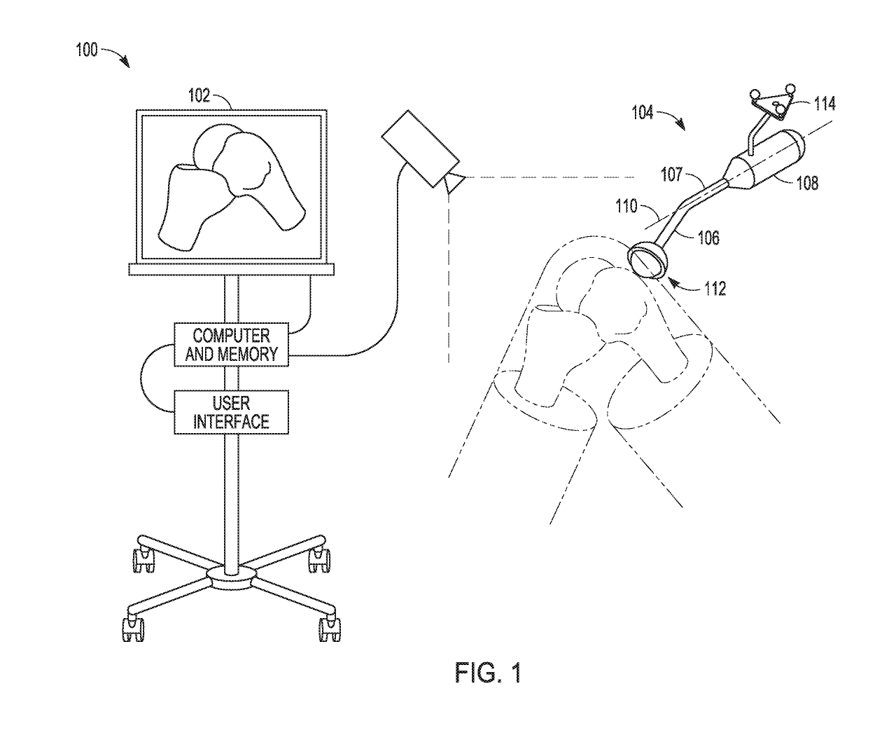

With initial reference to FIG. 1, a system 100 comprises a data collection unit 102 and a registration probe 104. The data collection unit 102and registration probe of the present teachings can be used in combination for anatomical registration prior to and during a surgical procedure.

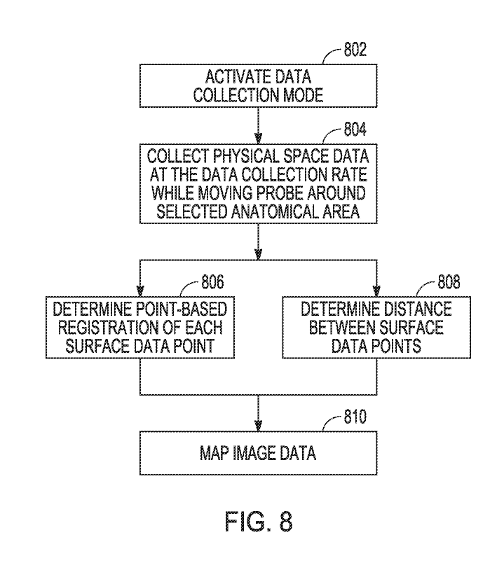

In one aspect, the data collection unit 102 can include, for example and without limitation, a computer assisted surgery system or a surgical navigation system. The data collection unit 102 can comprise, for example and without limitation, a camera, a vision system, and the like. The data collection unit 102 can have a data collection mode that can be selectively activated by a user via an activating mechanism. The data collection unit 102 can have a predefined data collection period for the data collection mode or can be selectively deactivated by a user via a termination mechanism. The data collection rate can be between about 1 Hz to about 60 Hz. As shown in FIG. 8, in operation and upon activation of the data collection mode (step 802), the data collection unit 102 can collect physical space data corresponding to each of a plurality of surface points on the selected anatomical area (step 804). The physical space data corresponding to each surface data point can be used to determine point-based registration (step 806). The physical space data corresponding to at least two sequential surface points can be used to determine the distance of travel of the registration probe 104 across the selected anatomical area and, thus, the distance between surface points (step 808). Next, the data collection unit maps image data describing the selected anatomical area using at least the point-based registration of each of the plurality of surface points (step 810).

Registration probes 104 according to the present teachings can have a distal portion 106, an intermediate portion 107, a proximal portion 108, and a longitudinal axis 110 defined by the intermediate portion 107. The registration probe can have an overall length of between about 100 mm to about 400 mm along its longitudinal axis 110. Furthermore, at least a portion of one of the distal portion 106 and the proximal portion 108 can be straight, curved, bent or offset from the registration probe longitudinal axis 110. Accordingly, the registration probe can be adapted to suit a wide variety of anatomical features as well as sizes and shapes of anatomical features.

In one aspect, the registration probe 104 can further comprise an engagement tip 112 operably coupled to the distal end of the distal portion 106. The engagement tip 112can comprise a dynamic engagement tip 112 and the dynamic engagement tip can further comprise a roller. The registration probe can also comprise a locator 114 coupled to the proximal portion 108. The dynamic engagement tip 112 can be rotatable with respect to the registration probe 104. In operation, the dynamic engagement tip 112 of the registration probe 104 can be placed in continuous and dynamic contact with and relative to a selected anatomical area. Dynamic contact can include moving the registration probe around the surface of the anatomical area. The data collection unit 102 can collect physical space data corresponding to individual surface points at a selected data collection rate during the selected time period that the data collection unit 102 is in data collection mode.

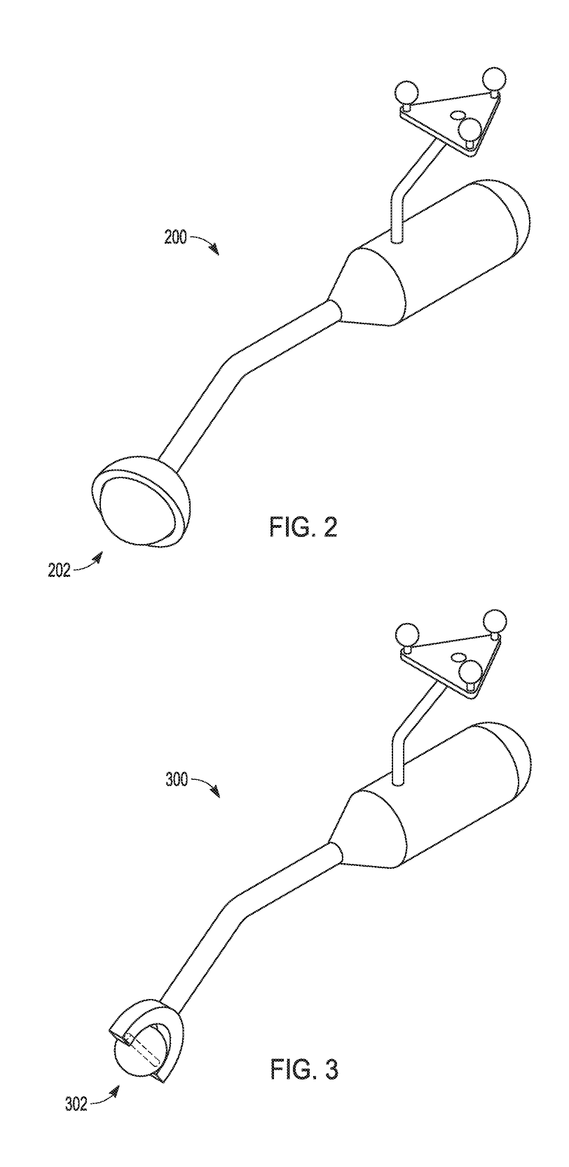

With reference to FIG. 2, the dynamic engagement tip of the registration probe 200 can comprise a radially unconstrained roller 202. The radially unconstrained roller 202 can move in any direction as it is rolled over the surface of the selected anatomical area. The diameter of the radially unconstrained roller 202 can be between about 5 mm to about 25 mm. Registration probe 200 can be used to facilitate registration of a wide variety of complex three-dimensional anatomic surfaces including their proximity to joints. Such complex three-dimensional anatomical surfaces can comprise at least portions of, for example and without limitation, the proximal and distal femur; the pelvis and acetabular region; the proximal tibia; the proximal humerus and scapula; the distal humerus and proximal radius; and the fibula, distal tibia, talus, and medial malleolus of the fibula. In operation, the data collection mode of the data collection unit 102 can be activated for a selected time period. During this time period, the radially unconstrained roller 202 can be maintained in contact with a selected anatomical area and the registration probe 200 can be moved around the selected anatomical area via the radially unconstrained roller 202 while the data collection unit 102 collects physical space data corresponding to individual surface points at the selected data collection rate. The data collection unit 102 can compensate for the shape and size of the radially unconstrained roller 202 along with the locator 114. The data collection unit 102 can also compute the distance traveled between physical space data corresponding to individual surface points.

With reference to FIG. 3, the dynamic engagement tip of the registration probe 300 can be a radially constrained roller 302. The roller 302 can be constrained to roll along a selected axis. The diameter of the radially constrained roller 302 can be between about 5 mm to about 25 mm. Registration probe 300 can be used for registering surfaces of a long bone that are aligned with the long axis of a long bone or that are transverse to the long axis of a long bone. Such anatomical surfaces can comprise at least portions of, for example and without limitation, circumferential anatomy of the proximal and distal femur, or proximal tibia, that is transverse or substantially transverse to the long axis. In operation, the data collection mode of the data collection unit 102 can be activated for a selected time period. During this time period, the radially constrained roller 302 can be maintained in contact with a selected anatomical area and the registration probe 300 can be moved along an axis relative to the selected anatomical area via rotation of the radially constrained roller 302 while the data collection unit 102 collects physical space data corresponding to individual surface points at the selected data collection rate. The data collection unit 102 can compensate for the shape and size of the radially constrained roller 302 along with the locator 114. The data collection unit 102 can also compute the distance traveled between individual surface points.

In another aspect, the data collection unit 102 can be, for example and without limitation, a computer assisted surgery system or a surgical navigation system. The data collection unit 102 can comprise, for example and without limitation, a camera, a vision system, and the like. The data collection unit 102 can have a data collection mode adapted for serial single point collection of data.

Here, the registration probe can further comprise an engagement tip 112 operably coupled to the distal end of the distal portion. The engagement tip can comprise a functional engagement tip 112 having a contact surface geometry that is complementary to a selected anatomical area. In operation, a data collection unit 102 can be activated and the functional engagement tip 112 placed into contact with a selected anatomical area. Upon placement, physical space data indicative of at least one of the size, location, and orientation of the functional engagement tip 112 can be recorded to facilitate registration of at least one of the size, location, and orientation of the selected anatomical area.

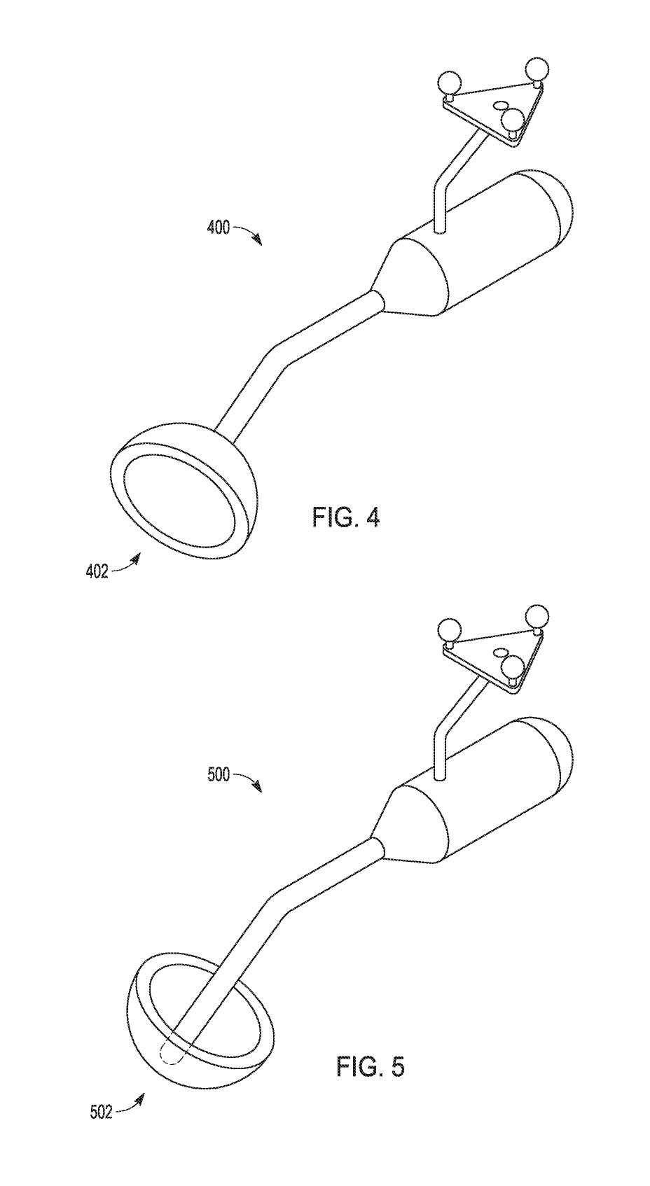

With respect to FIG. 4, the functional engagement tip 112 of the registration probe 400 comprises a convex contact surface 402. A registration probe 400 having a tip comprising a convex contact surface 402 can be used for registering the size, location, and orientation of concave anatomical features such as, for example and without limitation, the acetabulum.

With respect to FIG. 5, the functional engagement tip 112 of the registration probe 500 comprises a concave contact surface 502. A registration probe 500 having a tip comprising concave contact surface 502 can be used for registering the size, location, and orientation of convex anatomical features such as, for example and without limitation, the femoral head.

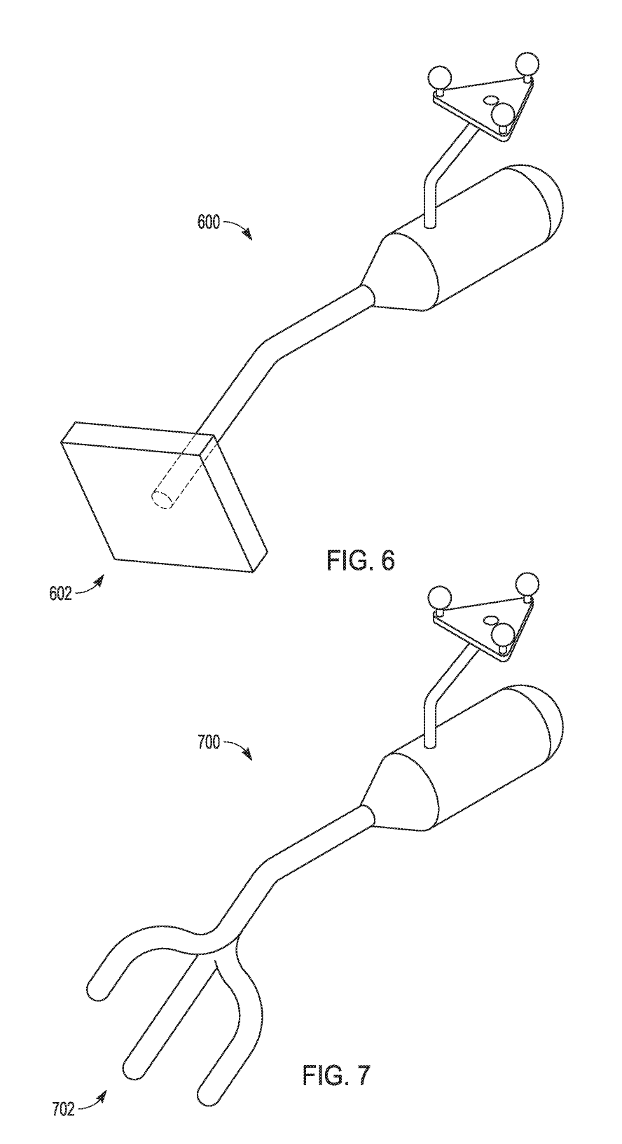

With respect to FIG. 6, the functional engagement tip 112 of the registration probe 600 comprises a planar contact surface 602. A registration probe 600 having a tip comprising a planar contact surface 602 can be used for registering planar features experienced or created during the process of joint replacement such as, for example and without limitation, the flat surfaces created by proximal tibial osteotomies, distal femoral osteotomies, anterior or posterior femoral osteotomies, and femoral chamfer osteotomies. In one operational example related to a hip joint, the femoral osteotomy plane can be recorded as well as the plane of the rim of the acetabulum.

With respect to FIG. 7, the functional engagement tip 112 of the registration probe 700 comprises a tip comprising an array of points 702. The tip comprising an array of points 702 can comprise at least three points. The array of points 702 can be used to register the orientation and location of a corresponding number of points on a complex anatomical surface simultaneously. The array of points 702 can be configured such that all points of the array of points 702 contact the selected anatomical surface in order to ensure accurate anatomical registration. A registration probe 700 having a tip comprising an array of points 702 can be used for registering a corresponding array of points on, for example and without limitation, the proximal femur, the distal femur, the proximal tibia, the pelvis, the acetabulum, the proximal humerous, the distal humerous, the proximal radius, and the proximal ulna. The array of points 702 can be defined with a circumscribed circle and, in a further aspect, the circumscribed circle can be between about 0.5 to about 3 inches in diameter.

In another aspect, the functional engagement tip 112 can be removably coupled to the distal portion 106 of the registration probe 104. Accordingly, a system comprising a registration probe 104 and a plurality of interchangeable functional engagement tips 112 is contemplated.

In yet other aspects, the data collection unit 102 can have a dynamic data collection mode as described with reference to FIGS. 2 and 3 and a single point collection mode as described with reference to FIGS. 4-7.

Some numbered examples of the present subject matter are listed below.

Example 1 can include or use a system comprising a registration probe. The registration probe can comprise a registration probe body having a distal portion, an intermediate portion, a proximal portion, and a longitudinal axis defined by the intermediate portion. The registration probe can also comprise a dynamic engagement tip operably coupled to a distal end of the distal portion. The dynamic engagement tip can be rotatable with respect to the registration probe. The registration probe can also have a locator coupled to the proximal portion of the registration probe body. In operation, the dynamic engagement tip of the registration probe can be placed in continuous contact with and moved around a selected anatomical area during anatomical registration.

Example 2 can be combined with the subject matter of Example 1 to include or use a data collection unit. The data collection unit can collect physical space data corresponding to a plurality of surface points at a data collection rate during a selected time period of anatomical registration. The data collection unit can determine at least one of a plurality of point-based registrations and distance between individual surface points.

Example 3 can be combined with the subject matter of Example 2 to include or use a data collection rate between about 1 to about 60 Hz.

Example 4 can include or use, or can optionally be combined with the subject matter of Examples 1-3, to include or use a dynamic engagement tip that comprises a roller.

Example 5 can be combined with the subject matter of Example 4 to include or use a radially unconstrained roller.

Example 6 can be combined with the subject matter of Example 4 to include or use a roller constrained to roll along a selected axis.

Example 7 can include or use, or can optionally be combined with the subject matter of Examples 1-6, to include or use a registration probe having an overall length of between about 100 mm to about 400 mm along the registration probe axis.

Example 8 can include or use, or can optionally be combined with the subject matter of Examples 1-7, to include or use a registration probe having a portion of one of the distal portion and the proximal portion that can be straight such that it extends along the registration probe axis.

Example 9 can include or use, or can optionally be combined with the subject matter of Examples 1-7, to include or use a registration probe having a portion of one of the distal portion and the proximal portion that can be curved relative to the registration probe axis.

Example 10 can include or use, or can optionally be combined with the subject matter of Examples 1-7, to include or use a registration probe having a portion of one of the distal portion and the proximal portion that can be bent relative to the registration probe axis.

Example 8 can include or use, or can optionally be combined with the subject matter of Examples 1-7, to include or use a registration probe having a portion of one of the distal portion and the proximal portion that can be offset from the registration probe axis.

Example 12 can include or use a device comprising a registration probe. The registration probe can have a distal portion, an intermediate portion, a proximal portion, and a longitudinal axis defined by the intermediate portion. The registration probe can also comprise a functional engagement tip coupled to a distal end of the distal portion. The functional engagement tip can have a contact surface geometry that is complementary to a selected anatomical area. The registration probe can also have a locator coupled to the proximal portion of the registration probe body. In operation, the functional engagement tip can facilitate registration of at least one of the size, location, and orientation of the selected anatomical area.

Example 13 can be combined with the subject matter of Example 12 to include or use a contact surface geometry of the functional engagement tip comprising a concave surface.

Example 14 can be combined with the subject matter of Example 12 to include or use, a contact surface geometry of the functional engagement tip comprising a convex surface.

Example 15 can be combined with the subject matter of Example 12 to include or use a contact surface geometry of the functional engagement tip comprising a planar surface.

Example 16 can be combined with the subject matter of Example 12 to include or use a functional engagement tip comprising a tip having an array of points.

Example 17 can be combined with the subject matter of Example 16 to include or use an array of points defined with a circumscribed circle.

Example 18 can be combined with the subject matter of Example 17 to include or use a circumscribed circle that can have a circumference of between about 0.5 to about 3 inches.

Example 19 can include or use, or can optionally be combined with the subject matter of Examples 12-18, to include or use a registration probe having an overall length of between about 100 to about 400 mm along the registration probe axis.

Example 20 can include or use, or can optionally be combined with the subject matter of Examples 12-19, to include or use a registration probe having a portion of one of the distal portion and the proximal portion that can be straight such that it extends along the registration probe axis.

Example 21 can include or use, or can optionally be combined with the subject matter of Examples 12-19, to include or use a registration probe having a portion of one of the distal portion and the proximal portion that can be curved relative to the registration probe axis.

Example 22 can include or use, or can optionally be combined with the subject matter of Examples 12-19, to include or use a registration probe having a portion of one of the distal portion and the proximal portion that can be bent relative to the registration probe axis.

Example 23 can include or use, or can optionally be combined with the subject matter of Examples 12-19, to include or use a registration probe having a portion of one of the distal portion and the proximal portion that can be offset from the registration probe axis.

Example 24 can include or use, or can optionally be combined with the subject matter of Examples 12-22, to include or use a functional engagement tip that can be removably coupled to the distal end of the distal portion.

Example 25 can include or use a method comprising activating a data collection unit for a selected time period; placing a dynamic engagement tip of a registration probe into contact with a selected anatomical area, wherein the registration probe has a distal portion, an intermediate portion, a proximal portion, and a longitudinal axis defined by the intermediate portion, wherein the dynamic engagement tip is operably coupled to a distal end of the distal portion, and wherein the dynamic engagement tip is rotatable with respect to the registration probe; moving the registration probe relative to the selected anatomical area via rotation of the dynamic engagement tip; and recording physical space data corresponding to a plurality of surface points at a data collection rate during the selected time period.

Example 26 can be combined with the subject matter of Example 25 to include or use a dynamic engagement tip comprising a roller.

Example 27 can be combined with the subject matter of Example 26 to include or use a radially unconstrained roller.

Example 28 can be combined with the subject matter of Example 26 to include or use a roller constrained to roll along a selected axis.

Example 29 can include or use, or can optionally be combined with the subject matter of Examples 25-28, to include or use a data collection rate between about 1 to about 60 Hz.

Example 25 can include or use a method comprising activating a data collection unit; placing a functional engagement tip of a registration probe into contact with a selected anatomical area, wherein the registration probe has a distal portion, an intermediate portion, a proximal portion, and a longitudinal axis defined by the intermediate portion, wherein the functional engagement tip is coupled to a distal end of the distal portion, and wherein the functional engagement tip comprises a contact surface geometry that is complementary to a selected anatomical area; and recording at least one of the size, location, and orientation of the functional engagement tip to facilitate registration of at least one of the size, location, and orientation of the selected anatomical area.

Example 26 can be combined with the subject matter of Example 25 to include or use a contact surface geometry of the functional engagement tip comprising a concave surface.

Example 27 can be combined with the subject matter of Example 25 to include or use, a contact surface geometry of the functional engagement tip comprising a convex surface.

Example 28 can be combined with the subject matter of Example 25 to include or use a contact surface geometry of the functional engagement tip comprising a planar surface.

Example 29 can be combined with the subject matter of Example 25 to include or use a functional engagement tip comprising a tip having an array of points.

Example 30 can be combined with the subject matter of Example 29 to include or use an array of points defined with a circumscribed circle.

Example 31 can be combined with the subject matter of Example 30 to include or use a circumscribed circle that can have a circumference of between about 0.5 to about 3 inches.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A system, comprising:
a registration probe, comprising:
a registration probe body having a distal portion, an intermediate portion, a proximal portion, and a longitudinal axis defined by the intermediate portion;
a dynamic engagement tip operably coupled to a distal end of the distal portion, wherein the dynamic engagement tip is rotatable with respect to the registration probe; and
a locator coupled to the proximal portion;
wherein, in operation, the dynamic engagement tip of the registration probe is placed in continuous contact with and moved around a selected anatomical area during anatomical registration.

2. The system of claim 1, further comprising a data collection unit, wherein the data collection unit collects physical space data corresponding to a plurality of surface points at a data collection rate during a selected time period of anatomical registration; wherein the data collection unit determines at least one of a plurality of point-based registrations and distance between individual surface points from the physical space data.

3. The system of claim 2, wherein the data collection rate is between about 1 to about 60 Hz.

4. The system of any one or combination of claims 1, wherein the dynamic engagement tip comprises a roller.

5. The system of claim 4, wherein the roller is radially unconstrained.

6. The system of claim 4, wherein the roller is constrained to roll along a selected axis.

7. The system of claim 1, wherein the registration probe has an overall length of between about 100 mm to about 400 mm along the registration probe axis.

8. The system of claim 1, wherein at least a portion of one of the distal portion and the proximal portion is straight such that it extends along the registration probe axis.

9. The system of claim 1, wherein at least a portion of one of the distal portion and the proximal portion is curved relative to the registration probe axis.

10. The system of claim 1, wherein at least a portion of one of the distal portion and the proximal portion is bent relative to the registration probe axis.

11. The system of claim 1, wherein at least a portion of one of the distal portion and the proximal portion is offset from the registration probe axis.

12. A device, comprising:
a registration probe having a distal portion, an intermediate portion, a proximal portion, and a longitudinal axis defined by the intermediate portion;
a functional engagement tip coupled to a distal end of the distal portion, wherein the functional engagement tip comprises a contact surface geometry that is complementary to a selected anatomical area;
a locator coupled to the proximal portion;
wherein, in operation, the functional engagement tip facilitates registration of at least one of the size, location, and orientation of the selected anatomical area.

13. The device of claim 12, wherein the contact surface geo of the functional engagement tip comprises a concave surface.

14. The device of claim 12, wherein the contact surface geometry of the functional engagement tip comprises a convex surface.

15. The device of claim 12, wherein the contact surface geometry of the functional engagement tip comprises a planar surface.

16. The device of claim 12, wherein the functional engagement tip comprises a tip having an array of points.

17. The device of claim 16, wherein the array of points are defined with a circumscribed circle.

18. The device of claim 17, wherein the circumscribed circle has a circumference of between about 0.5 to about 3 inches.

19. The device of claim 12, wherein the registration probe has an overall length of between about 100 to about 400 mm along the registration probe axis.

20. The device of claim 12, wherein at least a portion of one of the distal portion and the proximal portion is straight such that it extends along the registration probe axis.

21.-29. (canceled)
